(19) United States
(12) Patent Application Publication
(10) Pub. No.: US 2006/0252723 A1
(43) Pub. Date: Nov. 9, 2006

(54) Combined therapy utilizing reduction of DNA methyltransferase expression and/or activity and interferon

(75) Inventors: A. Robert MacLeod, Montreal (CA); Gregory Reid, Pointe-Claire (CA); Jeffrey Besterman, Bai D' Urfe (CA); Ernest Borden, Pepper Pike, OH (US); Frederic Reu, Shaker Heights, OH (US)

Correspondence Address: JOSEPH C. ZUCCHERO, SUITE 1200, 500 WEST CUMMINGS PARK, WOBURN, MA, 01801, US

(73) Assignee: Methylgene, Inc. The Cleveland Clinic Foundation

(21) Appl. No.: 11/393,380

(22) Filed: Mar. 30, 2006

Related U.S. Application Data
(60) Provisional application No. 60/667,285, filed on Apr. 1, 2005.

(51) Int. Cl. A61K 48/00; A61K 31/7072; A61K 31/53; A61K 31/513

(52) U.S. Cl. 514/44; 514/49; 514/242; 514/269

(57) ABSTRACT

The invention provides methods for the treatment of cancer comprising a reduction of DNA methyltransferase expression and/or activity and treatment and/or induction of interferon. The invention overcomes resistance of cancer cells to interferon

### RELATED ART

This application claims the benefit of U.S. Provisional Patent Application Ser. No. 60/667,285 filed on Apr. 1, 2005, the entire contents of which are incorporated by reference herein.

### BACKGROUND OF THE INVENTION

1. Field of the Invention

The invention relates to interferon-based treatment of cancer. More specifically, the invention relates to sensitizing cancer cells to interferon or overcoming interferon resistance of cancer cells. The invention provides methods for the treatment of cancer comprising a reduction of DNA methyltransferase expression and/or activity and treatment with and/or induction of interferon.

2. Summary of the Related Art

Bleumer, et al., Eur Urol, 44: 65-75 (2003) and Borden, Interferons. In: R. C. Bast, Kufe, D. W., Pollock, R. E., Weichselbaum, R. R., Holland, J. F., and Frei, E (ed.), Cancer medicine, 5 edition. Hamilton (Canada): BC Decker Inc. (2000) teach that IFN-α2 is the only modality of therapy that has been demonstrated in a randomized trial to increase survival of patients with metastatic renal cell carcinoma (RCC), albeit only for several weeks. Flanigan, R. C., Salmon, S. E., Blumenstein, B. A., Bearman, S. I., Roy, V., McGrath, P. C., Caton, J. R., Jr., Munshi, N., and Crawford, E. D. Nephrectomy followed by interferon alfa-2b compared with interferon alfa-2b alone for metastatic renal-cell cancer. N Engl J Med, 345: 1655-1659, 2001.

Flanigan et al., N Engl J Med, 345: 1655-1659 (2001) teaches that combined palliative resection of the primary and IFN therapy results in additional prolongation of survival. There is therefore a need for methods to enhance effectiveness of IFNs for RCC, to improve therapeutic outcomes for both primary and metastatic disease.

### BRIEF SUMMARY OF THE INVENTION

The invention provides methods for the treatment of cancer comprising a reduction of DNA methyltransferase expression and/or activity and treatment with interferon. The invention provides methods for sensitizing cancer cells to interferon or overcoming resistance of cancer cells to interferon.

Postulating that silencing of genes through methylation of their promoters is involved in resistance of renal cancer to IFNs, we determined the effect of two distinct pharmacological inhibitors of DNA methylation on IFN induced apoptosis. The nucleoside analogues 5-AZA-dC and 5-AZA-C, both small molecule inhibitors of DNMT, inhibit DNA methyltransferase activity once incorporated into DNA by covalently trapping the DNMT enzymes. As 5-AZA-dC and 5-AZA-C have pleiotropic effects, not attributed to demethylation, we have also used the potent and DNA methyltransferase 1 (DNMT1) selective antisense inhibitor MG98 (DNMT1 AS). The effect of DNMT1 depletion by either 5-AZA-dC or DNMT1 AS on IFN-induced apoptosis was evaluated in the IFN resistant human renal cancer cell line ACHN.

In a first aspect, the invention provides a method for sensitizing an interferon (IFN)-resistant cell to IFN-induced apoptosis. The method according to this aspect of the invention comprises demethylating a gene of the IFN-resistant cell effecting IFN resistance. In a preferred embodiment, the method comprises contacting the cell with at least one agent that reduces expression and/or activity of a DNA methyltransferase (DNMT), preferably DNA methyltransferase 1 (DNMT1). In a preferred embodiment the cell is a cancer cell.

In a second aspect, the invention provides a method of inducing apoptopsis in an IFN-resistant cell. The method according to this aspect of the invention comprises sensitizing the cell to IFN-induced apoptosis and contacting the cell with an IFN. In a preferred embodiment, the method comprises-demethylating a gene of the IFN-resistant-cell which effects IFN resistance, preferably by contacting the cell with an agent that reduces expression and/or activity of a DNMT, more preferably, DNMT1.

In a third aspect, the invention provides a method for treating a cancer patient having an IFN-resistant cancer cell. The method according to this aspect of the invention comprises sensitizing the IFN-resistant cancer cell to IFN-induced apoptosis and contacting the cell with a treatment effective amount of at least one IFN. In a preferred embodiment, the method comprises sensitizing the IFN-resistant cancer cell to IFN-induced apoptosis by demethylating a gene of the IFN-resistant cancer cell effecting IFN resistance, and contacting the cell with a treatment effective amount of an IFN. In another embodiment, demethylating a gene of the IFN-resistant cell effecting IFN resistance is effected by administering to the patient a treatment effective amount of an agent that reduces expression and/or activity of a DNA methyltransferase, more preferably DNMT1. In a preferred embodiment, the IFN-resistant cancer is renal cell carcinoma (RCC).

In preferred embodiments of the present invention, the IFN-resistant cancer cell is a human renal carcinoma cell. In preferred embodiments the human renal carcinoma cell is in a human body. In other preferred embodiments of the present invention the IFN-resistant cancer cell is a malignant melanoma cell, preferably in a human body.

In preferred embodiments of the present invention, the agent that reduces expression and/or activity of a DNMT is a small molecule inhibitor of DNMT and/or an antisense oligonucleotide complementary to DNMT mRNA.

The term “small molecule” as used in reference to the inhibition of DNMT is used to identify a compound having a molecular weight preferably less than 1000 Da, more preferably less than 800 Da, and most preferably less than 600 Da, which is capable of interacting with a DNMT and inhibiting the expression of a nucleic acid molecule encoding an DNMT isoform or activity of an DNMT protein. Inhibiting DNMT enzymatic activity means reducing the ability of a DNMT to add a methyl group to the C5 position of cytosine. In some preferred embodiments, such reduction of DNMT activity is at least about 50%, more preferably at least about 75%, and still more preferably at least about 90%. In other preferred embodiments, DNMT activity is reduced by at least 95% and more preferably by at least 99%.

### BRIEF DESCRIPTION OF THE DRAWINGS

FIG. 1 shows that DNMT1 protein is suppressed up to 48 hrs after the last MG98 (DNMT1 antisense) or 5-AZA-dC treatment.

FIGS. 2 A, B show that resistance to IFN-induced apoptosis is overcome by pretreatment with 5-AZA-dC and DNMT1 antisense (MG98), while treatment with mismatch control oligonucleotide (MM) or transfection reagent lipofectin did not (A). DNMT1 AS or MM alone did not result in significant apoptosis (B).

FIGS. 3 A-C show that IFN treatment increases caspase 3 activity only after pretreatment with DNMT1 inhibitors. Error bars (A, B) indicate standard deviation of fluorescence from duplicate wells. DNMT1 AS and 5-AZA-dC, but not MM, lipofectin, or media alone (ctrl) increased caspase 3 activity, which was further increased by IFN treatment only after DNMT1 depletion.

FIG. 4 shows that ACHN cells expressed stat1, stat2, and stat3 proteins, and this expression was not altered by DNMT1 depletion.

FIG. 5 shows that IFN and DNMT1 AS slow S phase and G2/M transition of ACHN cells.

FIGS. 6 A-D show that RASSF1A is silenced by promoter methylation in ACHN cells and that DNMT1 depletion leads to reactivation of RASSF1A expression with DNA demethylation.

FIGS. 7 A, B show that after pretreatment with DNMT1 antisense, interferon treatment results in increased RASSF1A protein.

### DETAILED DESCRIPTION OF THE PREFERRED EMBODIMENTS

The invention relates to interferon-based treatment of cancer. More specifically, the invention relates to methods of sensitizing cancer cells to interferon or overcoming interferon resistance of cancer cells. The invention provides methods for the treatment of cancer comprising a reduction of DNA methyltransferase expression and/or activity and treatment with and/or induction of interferon.

All patents and publications cited herein are hereby incorporated by reference in their entirety. In the case of any conflict between the teachings of the cited patents or publications and the present specification, such conflict shall be resolved in favor of the latter.

Postulating that silencing of genes through methylation of their promoters is involved in resistance of renal cancer to IFNs, we determined the effect of two distinct pharmacological inhibitors of DNA methylation on IFN induced apoptosis. The nucleoside analogues 5-AZA-dC and 5-AZA-C inhibit DNA methylation once incorporated into DNA by covalently trapping the DNMT enzymes. As 5-AZA-dC and 5-AZA-C have pleiotropic effects, not attributed to demethylation, we have also used the potent and DNMT1 selective antisense inhibitor MG98 (DNMT1 AS). The effect of DNMT1 depletion by either 5-AZA-dC or DNMT1 AS on IFN-induced apoptosis was evaluated in the IFN resistant human renal cancer cell line ACHN.

In a first aspect, the invention provides a method for sensitizing an interferon (IFN)-resistant cell to IFN-induced apoptosis. The method according to this aspect of the invention comprises demethylating a gene of the IFN-resistant cell effecting IFN resistance. In a preferred embodiment, the method comprises contacting the cell with at least one agent that reduces expression and/or activity of a DNA methyltransferase (DNMT), preferably DNA methyltransferase 1 (DNMT1). In a preferred embodiment, the cell is a cancer cell.

For purposes of the invention, the term “demethylating a gene” means causing at least one CpG dinucleotide within at least one gene to become non-methylated, or demethylated. Such demethylation of CpG dinucleotides within transcription control regions can cause such genes to be activated and expressed. For example, in the case of renal carcinoma cells that have become resistant to IFN-induced apoptosis, the present inventors have discovered that the RASSF1A gene has become methylated and deactivated. Demethylating the RASSF1A gene results in its reactivation and renders these cells sensitive to IFN-induced apoptosis.

Preferred methods for demethylating a gene of an IFN-resistant cancer cell include contacting the cell with a small molecule inhibitor of DNMT1 and/or an antisense oligonucleotide complementary to DNMT1 mRNA. Preferred methods for contacting a cell with an IFN comprise administering IFN-α and/or IFN-β to the cell or causing the cell to produce IFN-α and/or IFN-β by administering an IFN-α and/or IFN-β inducing agent. Preferred IFN-α and/or IFN-β inducing agents include PolyI:C, double-stranded RNA and immunostimulatory agents. In preferred embodiments, demethylation of a gene precedes contacting the cell to IFN. Preferably, demethylation is carried out by administration of a demethylating agent from about 4 hours to about 8 days before exposure to IFN.

In a second aspect, the invention provides a method of inducing apoptosis in an IFN-resistant cell. The method according to this aspect of the invention comprises sensitizing the cell to IFN-induced apoptosis and contacting the cell with an IFN. In a preferred embodiment, the method comprises demethylating a gene of the IFN-resistant cell which effects IFN resistance, preferably by contacting the cell with an agent that reduces expression and/or activity of a DNMT, more preferably, DNMT1.

In a third aspect, the invention provides a method for treating a cancer patient having an IFN-resistant cancer cell. The method according to this aspect of the invention comprises sensitizing the IFN-resistant cancer cell to IFN-induced apoptosis and contacting the cell with a treatment effective amount of at least one IFN. In a preferred embodiment, the method comprises sensitizing the IFN-resistant cancer cell to IFN-induced apoptosis by demethylating a gene of the IFN-resistant cancer cell effecting IFN resistance, and contacting the cell with a treatment effective amount of an IFN. In a preferred embodiment, demethylating a gene of the IFN-resistant cell effecting IFN resistance is effected by administering to the patient a treatment effective amount of an agent that reduces expression and/or activity of a DNA methyltransferase, more preferably DNMT1. In a preferred embodiment, the IFN-resistant cancer is renal cell carcinoma (RCC). The term “demethylating a gene” is intended to have the same meaning as for the first aspect of the invention.

Preferred methods for demethylating a gene of an IFN-resistant cancer cell in a patient include administering to the patient having at least one IFN-resistant cancer cell a small molecule inhibitor of DNMT1 and/or an antisense oligonucleotide complementary to DNMT1 mRNA. Inhibitors of DNMT1 enzyme are known in the art. (See e.g., U.S. Pat. No. 6,268,137 and U.S. Patent publication no. 20030096777) Antisense oligonucleotides that inhibit DNA methyltransferase expression are also known in the art (see e.g., U.S. Pat. Nos. 6,066,625; 6,184,211; 6,020,318; 5,578,716; 5,919,772; 6,506,735; 6,221,849 and 6,054,439) and include oligonucleotides currently in human clinical trials.

Small molecule inhibitors include, but are not limited to, inhibitors of DNA methlytransferase enzyme having the general structure: 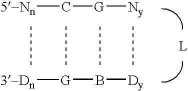
wherein each N is independently any nucleotide, n is a number from 0-20, C is 5-methylcytidine, G is guanidine, y is a number from 0-20, L is a linker, each D is a nucleotide that is complementary to an N such that Watson-Crick base pairing takes place between that D and the N such that the Nₙ—C-G-N_{y} and the Dₙ-G-B-D_{y} form a double helix, B is cytosine, inosine, uridine, 5-bromocytosine or 5-fluorocytosine, or abasic deoxyribose, the linkage between B and G is a phosphorothioate or phosphorodithioate linkage, dotted lines between nucleotides represent hydrogen bonding between the nucleotides, and the total number of nucleotides ranges from about 10 to about 50.

Such inhibitors also include, but are not limited to, inhibitors of DNA methyltransferase enzyme having the general structure:

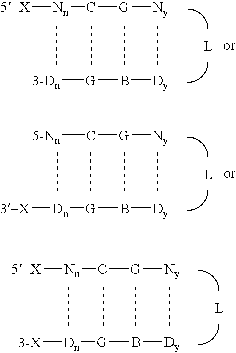

wherein each N is independently any nucleotide, n is a number from 0-20, C is 5-methylcytidine, G is guanidine, y is a number from 0-20, L is a linker, each D is a nucleotide that is complementary to an N such that Watson-Crick base pairing takes place between that D and the N such that the Nₙ—C-G-N_{y} and the Dₙ-G-B-D_{y} form a double helix, B is cytosine, inosine, uridine, 5-bromocytosine, abasic deoxyribose, or 5-fluorocytosine, dotted lines between nucleotides represent hydrogen bonding between the nucleotides, B and G are linked by a phosphorothioate or phosphorodithioate linkage and the total number of nucleotides ranges from about 10 to about 50, X is an antisense oligonucleotide of from about 10 to about 50 nucleotides in length, which is complementary to a portion of a mRNA encoding DNA methyltransferase enzyme, and L can optionally be X.

In accordance with an aspect of the present invention, a small molecule inhibitor may also include, but is not limited to, 5-aza-cytidine (5-AZA-C), 5-aza-deoxycytidine (5-AZA-dC), 5-flouro-2′-deoxycytidine, 5,6-dihydro-5-azacytidine and Zebularine.

Preferred non-limiting examples of antisense oligonucleotides complementary to mRNA or double-stranded DNA encoding DNMT in accordance with an aspect of the present invention, and which inhibit DNA methyltransferase expression include, but are not limited to, those presented in Table 1.

TABLE 1

| | |
|---|---|
| SEQ ID NO. | SEQUENCE |

| | |
|---|---|
| 1 | 5′-AAG CAT GAG CAC CGT TCT CC-3′ |
| 2 | 5′-TTC ATG TCA GCC AAG GCC AC-3′ |
| 3 | 5′-GCT GTC TCT TTC CAA ATC TT-3′ |
| 4 | 5′-TTT CTG TTA AGC TGT CTC TT-3′ |
| 5 | 5′-TTC TCC TTC ACA CAT TCC TT-3′ |
| 6 | 5′-CGT GCA AGA GAT TCA ATT TC-3′ |
| 7 | 5′-AAG TCA CAT AAC TGA TTC TT-3′ |
| 8 | 5′-CTC GGA TAA TTC TTC TTT AC-3′ |
| 9 | 5′-CCA GGT AGC CCT CCT CGG AT-3′ |
| 10 | 5′-AGG GAT TTG ACT TTA GCC AG-3′ |
| 11 | 5′-TCC AAG GAC AAA TCT TTA TT-3′ |
| 12 | 5′-CAT GAG CAC CGT TCT CCA AG-3′ |
| 13 | 5′-ACG TCC ATT CAC TTC CCG GT-3′ |
| 14 | 5′-TCA CTT CTT GCT TGC TTC CC-3′ |
| 15 | 5′-GCT TGG TTC CCG TTT TCT AG-3′ |
| 16 | 5′-CTA GAC GTC CAT TCA CTT CC-3′ |
| 17 | 5′-ACT CTA CGG GCT TCA CTT CT-3′ |
| 18 | 5′-TCT GCC ATT CCC ACT CTA CG-3′ |
| 19 | 5′-CAT CTG CCA TTC CCA CTC TA-3′ |
| 20 | 5′-GGC ATC TGC CAT TCC CAC TC-3′ |
| 21 | 5′-ATC GGA CTT GCT CCT CCT GG-3′ |
| 22 | 5′-GGT GAC GGG AGG GCA GAA CT-3′ |
| 23 | 5′-TGC CAG AAA CAG GGG TGA CG-3′ |
| 24 | 5′-GTG CAT GTT GGG GAT TCC TG-3′ |
| 25 | 5′-GTG AAC GGA CAG ATT GAC AT-3′ |
| 26 | 5′-AGG CCA CAA ACA CCA TGT AC-3′ |
| 27 | 5′-CGA ACC TCA CAC AAC AGC TT-3′ |
| 28 | 5′-GAT AAG CGA ACC TCA CAC AA-3′ |
| 29 | 5′-CTG CAC AAT TTG ATC ACT AA-3′ |
| 30 | 5′-CAG AAA CAG GGG TGA CGG GA-3′ |
| 31 | 5′-GCA CAA AGT ACT GCA CAA TT-3′ |
| 32 | 5′-TCC AGA ATG CAC AAA GTA CT-3′ |
| 33 | 5′-CCA AGG CCA CAA ACA CCA TG-3′ |
| 34 | 5′-CCA GGT AGC CCT CCT CGG AU-3′ |
| 35 | 5′-AAG CAT GAG CAC CGT TCU CC-3′ |
| 36 | 5′-UUC ATG TCA GCC AAG GCC AC-3′ |
| 37 | 5′-CGA ACC TCA CAC AAC AGC UU-3′ |
| 38 | 5′-GAU AAG CGA ACC TCA CAC AA-3′ |
| 39 | 5′-CCA AGG CCA CAA ACA CCA UG-3′ |

Preferred oligonucleotides have nucleotide sequences of from about 13 to about 35 nucleotides. Additional preferred oligonucleotides have nucleotide sequences of from about 20 to about 35 nucleotides. Yet additional preferred oligonucleotides have nucleotide sequences of from about 13 to about 19 nucleotides.

Particularly preferred antisense oligonucleotides according to this aspect of the invention include chimeric oligonucleotides and hybrid oligonucleotides.

For purposes of the invention, a “chimeric oligonucleotide” refers to an oligonucleotide having more than one type of internucleoside linkage. One preferred embodiment of such a chimeric oligonucleotide is a chimeric oligonucleotide comprising a phosphorothioate, phosphodiester or phosphorodithioate region, preferably comprising from about 2 to about 12 nucleotides, and an alkylphosphonate or alkylphosphonothioate region. Preferably, such chimeric oligonucleotides contain at least three-consecutive internucleoside linkages selected from phosphodiester and phosphorothioate linkages, or combinations thereof.

For purposes of the invention, a “hybrid oligonucleotide” refers to an oligonucleotide having more than one type of nucleoside. One preferred embodiment of such a hybrid oligonucleotide comprises a ribonucleotide or 2′-O-substituted ribonucleotide region, preferably comprising from about 2 to about 12 2′-O-substituted nucleotides, and a deoxyribonucleotide region. Preferably, such a hybrid oligonucleotide will contain at least three consecutive deoxyribonucleosides and will also contain ribonucleosides, 2′-O-substituted ribonucleosides, or combinations thereof. In a preferred embodiment, the deoxynucleotide region is flanked on either side by a 2′-O-substituted region. In one particularly preferred embodiment, the 2′-O-substituted regions are 2′-O-methyl regions, most preferably having four 2′-O-methyl nucleosides. In certain preferred embodiments the entire backbone of the oligonucleotide is a phosphorothioate backbone.

The exact nucleotide sequence and chemical structure of an antisense oligonucleotide according to the invention can be varied, so long as the oligonucleotide retains its ability to inhibit DNMT expression at a high level of efficacy. This is readily determined by testing whether the particular antisense oligonucleotide is active in a DNMT mRNA assay, DNMT enzyme assay, a soft agar growth assay, or an in vivo tumor growth assay, all of which are known in the art.

Preferred methods for exposing a cell to an IFN comprise administering to a patient IFN-α and/or IFN-β, or by administering to the patient an IFN-α and/or IFN-β inducing agent. An IFN-inducing agent is an agent that causes an immune cell to produce IFN. Preferred IFN-α and/or IFN-β inducing agents include polyI:C, double-stranded RNA, and immunostimulatory oligonucleotides. The IFN-inducing agent may cause the cell to produce an endogenous or exogenous (i.e., recombinant) IFN.

In preferred embodiments of an aspect of the present invention the IFN-resistant cancer cell is a human renal carcinoma cell. In preferred embodiments of an aspect of the present invention the human renal carcinoma cell is in a human body.

In a preferred embodiment, patients are assigned to one of two schedules of MG98. In one group, patients are treated with a 2-hour IV infusion of MG98 given twice per week for three weeks followed by one week of rest. This is administered with a fixed dose of INF administered subcutaneously three times per week over the full course of treatment. In the second group, patients receive MG98 in two 7-day continuous IV infusions, each followed by one week of rest and combined with the same dose and schedule of INF. Both 4-week regimens constitute 1 cycle of treatment. In a preferred embodiment, patients are randomized with equal probability either to treatment with the recommended dose and schedule of MG98 combined with INF or to treatment with the same dose and schedule of INF administered as a single agent. Preferably, MG98 is administered at a dose of from about 80 mg/m²/day to about 200 mg/m²/day. For continuous infusion, MG98 is administered at a starting dose of about 125 mg/m²/day. For intermittent administration, preferably a starting dose of about 160 mg/m²/day of MG98 is used. In preferred embodiments, IFN is administered at a weekly total dose of between about 25 to 45 MIU. Preferably, the starting dose of INF administered in combination with MG98 or as monotherapy is about 12 MIU/m²/day given subcutaneously three times per week throughout the course of treatment.

The following examples are intended to further illustrate certain particularly preferred embodiments of the invention, and are not intended to limit its scope.

### EXAMPLE 1

### Inhibition of DNA Methylation

ACHN Renal cell carcinoma cells were cultured at 37° C. in 5% CO₂ using Minimum Essential Medium (GIBCO, Invitrogen, Carlsbad, Calif.) with 0.1 mM non-essential amino acids (GIBCO), 1.0 mM pyruvate (GIBCO), 10% fetal bovine serum, penicillin G (50 U/ml), and streptomycin (50 μg/ml). WM9 melanoma cells (13) were grown in RPMI medium (GIBCO) containing 10% fetal bovine serum, penicillin G (50 U/ml), and streptomycin (50 μg/ml) under the same incubator conditions.

To selectively downregulate DNMT1, cells were transfected with MG98 (MethylGene, Quebec, Canada), a second-generation 4×4 2′methyl phosphorothioate oligonucleotide antisense against the 3′ UTR of DNMT1 mRNA (5′-UUCATGTCAGCCAAGGCCAC-3) or mismatch (underlined) control oligonucleotide (5′-TTAATGTAACCTAAGGTCAA-3′) (SEQ ID NO:40) at indicated concentrations starting one day after plating at 15000 cells/cm². Transfections were performed daily with 6.25 μg/ml Lipofectin (Invitrogen, Carlsbad, Calif.) in OptiMem (GIBCO, Invitrogen, Carlsbad, Calif.) over 4 hr. Before and after transfections cells were washed once with PBS. Every second day cells were replated at 15000 cells/cm² four hr after the preceding transfection. 5-AZA-dC (Sigma-Aldrich, St Louis, Mo.) stock solution (100 mM) in DMSO and working solutions for one time use in PBS (1 mM) were stored at −20° C. Daily treatments were performed with freshly thawed working solution diluted in complete media to indicated concentrations. Plating conditions were the same as for transfections and cells were replated 4 hr after 5-AZA-dC treatment every second day into complete media not containing 5-AZA-dC.

Twenty to 40 μg protein from whole cell lysates were probed for DNMT1 by polyclonal antibody (pAB) (MethylGene, Quebec, Canada) and actin by mAB (Sigma-Aldrich, St Louis, Mo.) after separation in 8-12% SDS-polyacrylamide gels and transfer to PVD membranes. For detection of bound primary antibody, PVD membranes were incubated with horseradish tagged goat anti-mouse antibody (Bio-Rad, Hercules, Calif.), followed after washing with TBST, by staining with enhanced chemiluminescence solution (Amersham, Piscataway, N.J.).

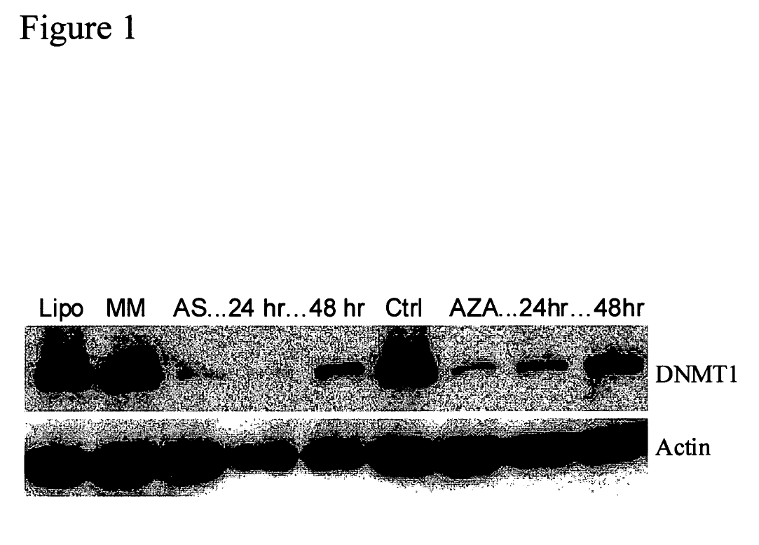

DNMT1 protein is suppressed up to 48 hrs after the last AS or 5-AZA-dC treatment. ACHN cells were transfected daily over 9 days with 40 nM DNMT1 AS (AS) or treated daily with 200 nM 5-AZA-dC (AZA) over 4 days. Protein was isolated 4, 24, and 48 hrs after the last treatment. Untreated cells (Ctrl), lipofectin only (Lipo), and mismatch (MM) treated cells served as controls. (See FIG. 1). Similar DNMT1 reduction was observed in more than three independent experiments.

### EXAMPLE 2

### Effect of Demethylation and IFN Treatment on Other Gene Expression

Cells were treated as described in Example 1. RNA was isolated using the Trizol (Invitrogen, Carlsbad, Calif.) method and cDNA prepared with a superscript III first strand synthesis kit including a final Rnase H digestion step (Invitrogen) according to the manufacturer's instructions.

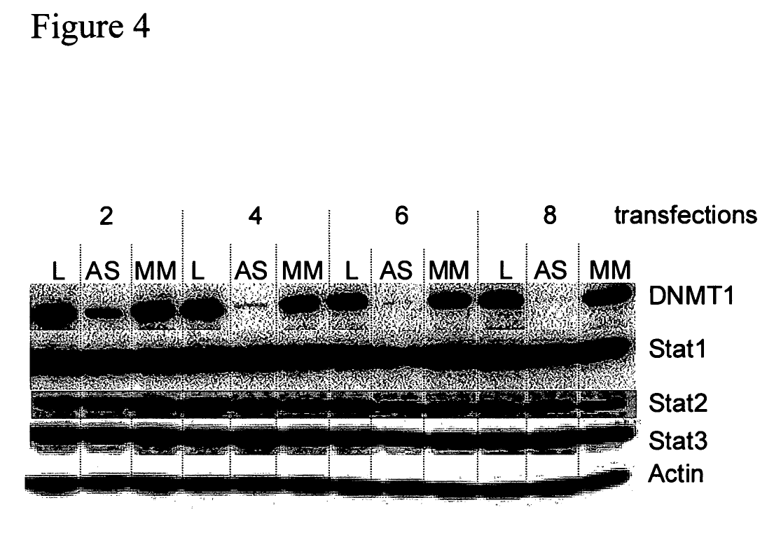

At baseline ACHN cells expressed stat1, stat2, and stat3 proteins, and this expression was not altered by DNMT1 depletion (FIG. 4).

Using taqman primers (Applied Biosystems, Foster City, Calif.) real-time RT-PCR was performed according to the manufacturer instructions for stat1, stat2, and stat3 genes using ABI PRISM Sequence Detection Instrument 7700 (Applied Biosystems). These experiments revealed no increase in stat1 or stat2 expression levels upon DNMT1 AS treatment (0.27, 1.05 fold change over untreated, respectively) while in mismatched control treated cells 50 U/ml IFN-β over 16 hr increased stat1 and stat2 (2.24 and 6.94 fold, respectively) confirming western blot results and suggesting that DNMT1 AS did not induce endogenous IFNs. 5-AZA-dC only minimally affected expression of stat1 and stat2 (0.57 and 1.38 fold change over untreated, respectively), as assessed by real-time RT-PCR.

To identify additional genes reactivated through DNA demethylation and of potential importance for the sensitization to IFN-induced apoptosis we performed cDNA array analysis with RNA harvested from ACHN cells 24 hr after the 8th DNMT1 AS transfection and 16 hr after 50 U/ml IFN β 1a treatment. RNA was harvested as mentioned above using Trizol (Invitrogen, Carlsbad, Calif.) method followed by transcription into cRNA, according to Affymetrix (Santa Clara, Calif.) recommendations for U133A array hybridization. Expression was analyzed using Affymetrix software.

Compared to mismatch oligonucleotide, DNMT1 AS treatment led to a 94% reduction in DNMT1 expression without affecting expression of other DNMTs (data not shown). Expression of genes known to be involved in IFN-induced apoptosis was not significantly altered by DNMT1 AS treatment (Table 2 below) and DNMT1 AS only increased one known IFN-stimulated gene of unknown function (IF127) significantly (p<0.045) at least two-fold over MM treated cells.

TABLE 2

| | | CpG island closest to translation start (=+1) | | | | Fold change in expression | | | |
|---|---|---|---|---|---|---|---|---|---|
| | | | | | obs/exp | | MM + | AS + | AS + |
| Reference ID | Gene name | Location | Length | % GC | CpG | AS/MM | IFNβ/MM | IFNβ/AS | IFNβ/MM + IFNβ |
| NM_007315 | Stat1 | −4646 to −3676 | 971 | 61.5 | 0.891 | 0.60 | 1.42 | 2.22 | 0.90 (NS) |
| NM_005419 | Stat2 | −1009 to −841 | 224 | 54.4 | 0.601 | 0.68 (NS) | 2.14 | 4.17 | 1.13 (NS) |
| NM_003810 | TNFSF10 | −2306 to −1947 | 358 | 50.5 | 0.707 | 0.93 (NS) | 9.38 | 10.06 | 1.28 (NS) |
| | (TRAIL/Apo2L) | | | | | | | | |
| NM_017523 | HSXIAPAF1 | −1458 to −1251 | 208 | 50.9 | 0.6 | 3.36 (NS) | 35.02 | 7.78 | ‘1.8 (NS) |
| | (XAF1) | | | | | | | | |
| NM_001225 | Caspase 4 | >−10 000 | | | | 1.41 (MI) | 1.93 | 1.31 (MI) | 1.13 (NS) |
| NM_002198 | IRF1 | −1720 to −526 | 1195 | 73.2 | 0.894 | 1.32 (NS) | 1.82 | 1.73 | 1.22 (NS) |
| NM_002534 | OAS1 | >−10 000 | | | | 0.80 (NS) | 18.77 | 18.51 | 0.86 (NS) |
| NM_002759 | PRKR | −4336 to −3922 | 415 | 51.3 | 0.952 | 0.71 | 1.72 | 2.73 | 1.12 (NS) |
| | (PKR) | | | | | | | | |

One hundred and thirty-seven genes were increased at least two-fold by DNMT1 AS alone compared to the mismatch oligonucleotide (data not shown).

### EXAMPLE 3

### Cell Cycle Analysis

To gain some insight into which genes to focus, a cell cycle analysis was undertaken using propidium iodide staining of nuclei. Cells were trypsinized at the indicated time points, washed once with PBS, then stained with one ml PI staining solution (0.0125 g/L propidium iodide, 0.25 g/L sodium citrate, 0.25 ml/L triton×100 in distilled water) on ice and protected from light over 2 hr to stain nuclear DNA. Analysis was performed by flow cytometry using Modfit software (Verity software house, Topsham, ME).

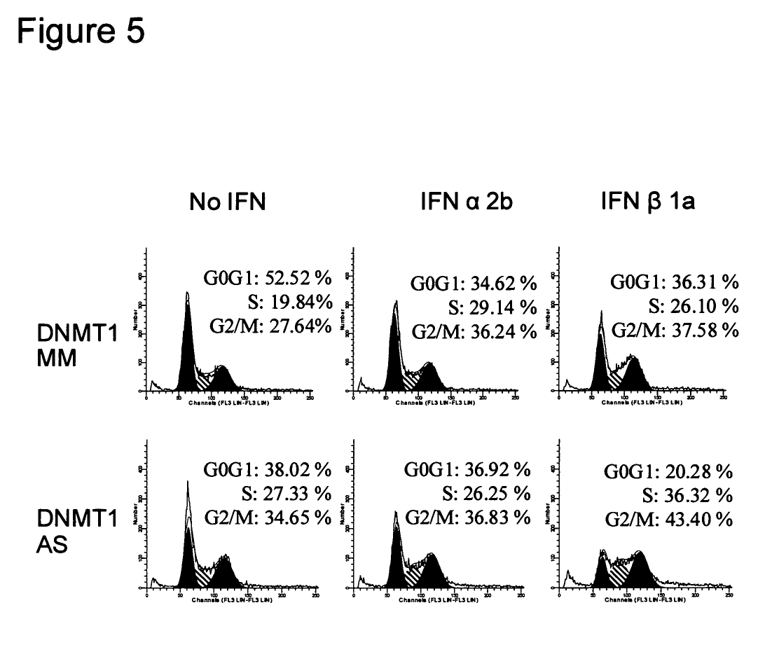

Treatment of ACHN cells with IFN α or IFN β (50 U/ml over 48 hr) led to accumulation of cells in S phase and G2/M 48 hr after IFN treatment (FIG. 5). A similar effect was observed after DNMT1 AS treatment alone (FIG. 5). Growth was reduced with either IFN or DNMT1 AS treatment (data not shown) suggesting transition to G1 was blocked rather then mitosis increased. IFN β and DNMT1 AS treatment combined led to most pronounced inhibition of transition into G1 (less than 50% compared to mismatch oligonucleotide treatment alone, FIG. 5). Thus, IFN and DNMT1 AS resulted in slower transition of ACHN cells through S phase and G2/M which suggested genes that affect this compartment may be involved in the response to IFNs.

### EXAMPLE 4

### DNMT1 Depletion Leads to Demethylation and Reactivation of RASSF1A

RASSF1A inhibits the anaphase-promoting complex/cyclosome (APC) in prometaphase and overexpression can arrest cells in prometaphase. Knowing that RASSF1A carries a hypermethylated promoter in up to 91% of clinical renal cancer specimens and 100% of renal cancer cell lines evaluated, including ACHN, we evaluated the expression of RASSF1A upon selective depletion of DNMT1 in ACHN cells. Cells were treated as described in Example 1.

One microgram of genomic DNA, harvested with a blood DNA mini kit (Quiagen, Valencia, Calif.), was used for bisulfite modification with the CpGenome kit (Chemicon International, Temecula, Calif.) according to the manufacturer's instructions with final resuspension in 20 μl of 10 mM Tris/Ci, pH 8.5. Four μl of bisulfite modified DNA was used per 25 μl MSP reaction. Primers for RASSF1A MSP as published (15) were (5′ to 3′): M forward: GGG TTT TGC GAG AGC GCG (SEQ ID NO:41), M reverse: GCC AAG CGC AAA CAA TCG (SEQ ID NO:42), U forward: GGT TIT GTG AGA GTG TGT TTA G (SEQ ID NO:43), U reverse: AAA CCA AAC ACA AAC AAT CAC (SEQ ID NO:44). PCR settings for methylated (M) primer pair were denaturation at 95° C. for 5 min, followed by 35 cycles with a 1 min denaturation step, 30 sec annealing at 60° C., and extension at 72° C. for 30 sec. Final extension after 35 cycles was at 72° C. for 4 min. For sequences specific for unmethylated (U) DNA settings were the same except for annealing at 55° C.

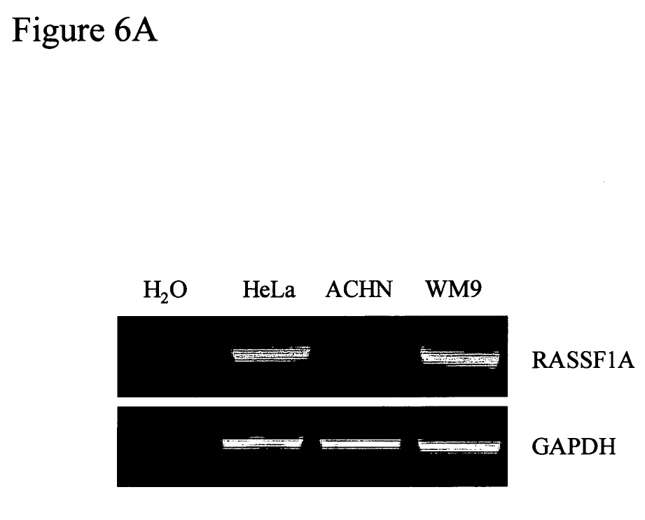

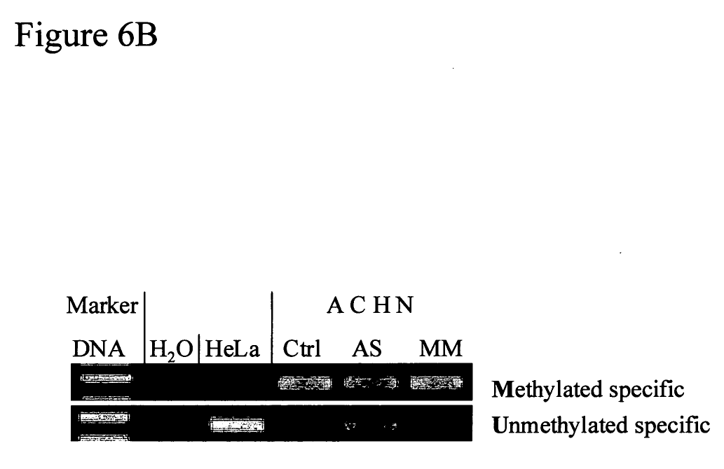

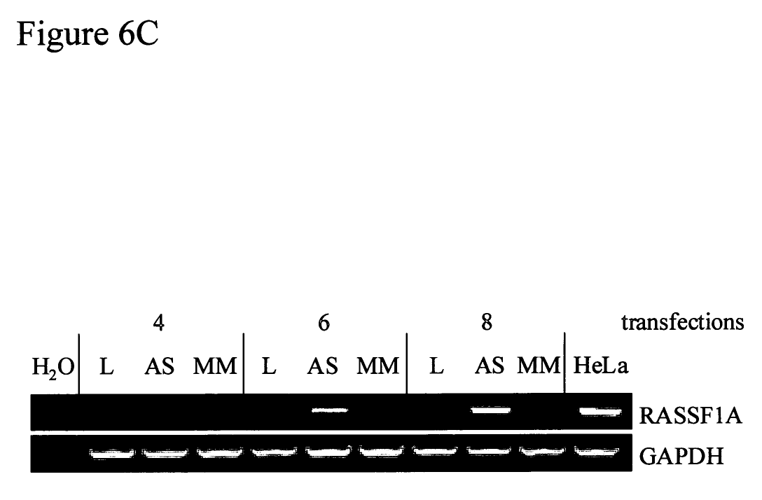

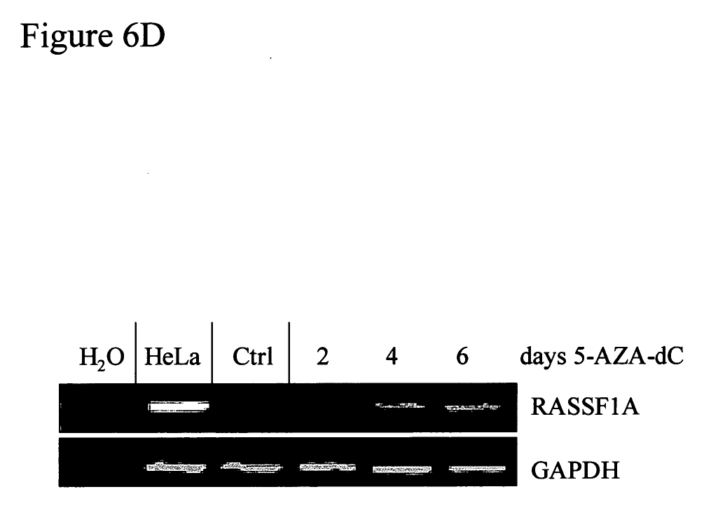

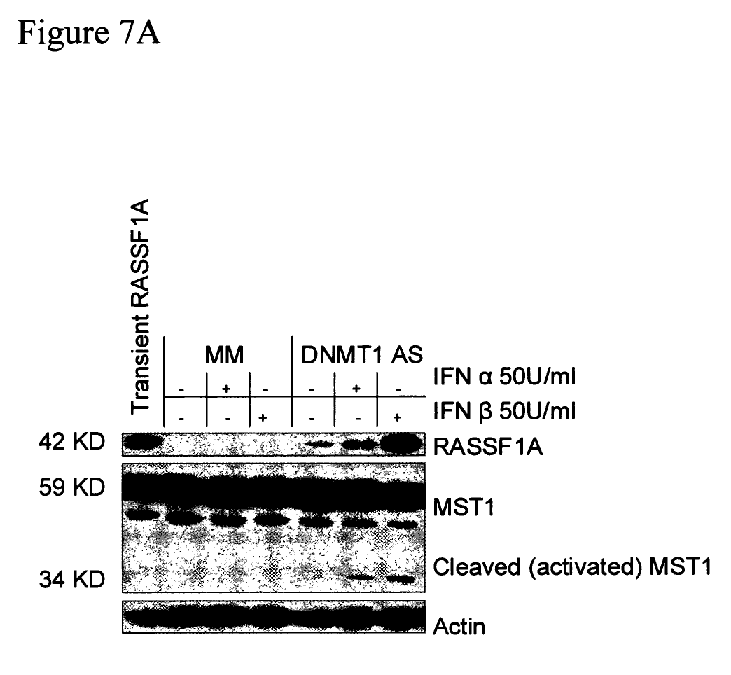

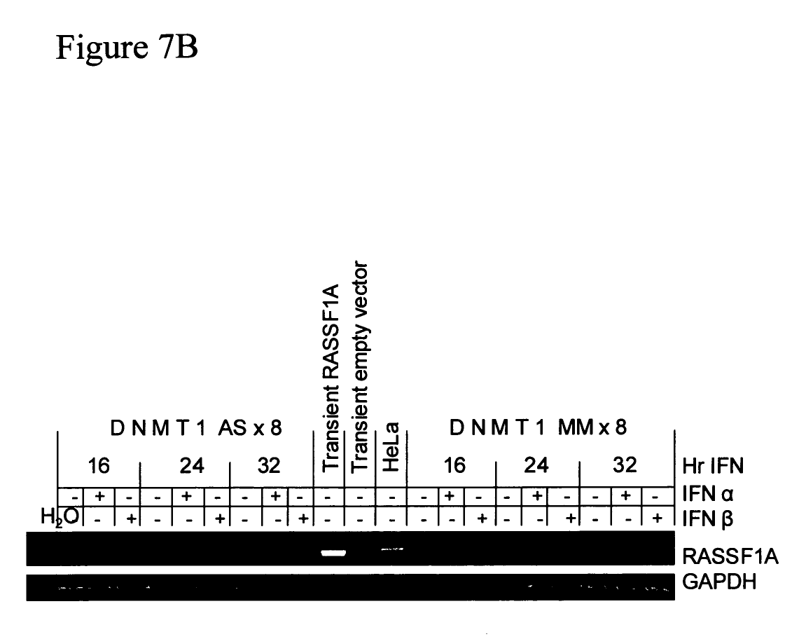

DNMT1 depletion was effective at demethylating the promoter region and reactivating methylation-silenced message of RASSF1A in ACHN cells (FIG. 6B-D). Additionally, treatment with IFNs after DNMT1 AS but not MM pretreatment led to increase in RASSF1A protein expression, more pronounced with IFN-β than -α, without effect on transcription suggesting posttranscriptional regulation (FIGS. 7A, B).

To assess reactivation, RT-PCR was performed with primers that amplified RASSF1 variants regulated by a promoter that has been described as hypermethylated in cancer. Primers were 5′-AGC GTG CCA ACG CGC TGC GCA T-3′ (sense) (SEQ ID NO:45) and 5′-CAG GCT CGT CCA CGT TCG TGT C-3′ (antisense) (SEQ ID NO:46). Settings used were 95° C.-4 min, (95° C.-1 min, 52° C.-30 sec, 72° C.-30 sec for 30 to 35 cycles), 72° C.-4 min. GAPDH was amplified with the settings 95° C.-4 min, (95° C.-45 sec, 55° C.-30 sec, 72° C.-50 sec for 15 to 25 cycles), 72° C.-4 min. GAPDH primers were 5′-CAG ACC TAC TCA GGG ATT C-3′ (sense) (SEQ ID NO:47) and 5′-GAG CCA GAC GCT GCT TTG T-3′ (antisense) (SEQ ID NO:48). For sequencing of full length RASSF1 cDNA RT-PCR with primers (5′ to 3′) CGC CCA GTC TGG ATC CTG (sense) (SEQ ID NO:49) and CTC AAT GCC TGC CTT ATT CTG (antisense) (SEQ ID NO:50) was performed using proofreading platinum Pfx polymerase (Invitrogen, Carlsbad, Calif.) and the following settings: Denaturation at 95° C. for 4 min followed by 30 cycles of denaturation at 95° C. for 45 sec, annealing at 58° C. for 30 sec, and extension at 68° C. for 3 min followed by final extension at 68° C. for 8 min. Products were cloned into Zero Blunt cloning vector for sequencing and then into pcDNA3.1 for overexpression. RT-PCR demonstrated that RASSF1A was not expressed in ACHN cells while WM9 cells, which are known to undergo apoptosis in response to IFN-β, expressed RASSF1A (FIG. 6A).

Methylation specific PCR was used to confirm hypermethylation in the RASSF1A promoter region. Treatment with DNMT1 AS led to demethylation (FIG. 6B). Both DNMT1 inhibitors reactivated transcription of RASSF1A (FIGS. 6C, D). Amplification of full length RASSF1A cDNA after AS treatment yielded a single band in AS treated cells, and no band in MM treated or native ACHN controls (data not shown). The band from AS treated ACHN cells was cloned for sequencing. Four independent clones were sequenced, all revealed RASSF1A, NM_{—}007182, with a single nucleotide polymorphism at nucleotide 528 (T instead of G) leading to a conservative change at amino acid position 133 (serine for alanine).

### EXAMPLE 5

### Overexpression of RASSF1A

Attempting to assess the role RASSF1A reactivation (by DNMT1 depletion) plays in sensitization to IFN, RASSF1A cDNA from DNMT1 AS treated ACHN cells was overexpressed (pcDNA3.1) in native ACHN cells. Transfection for overexpression was performed using 1 μg/ml plasmid and 6 μl/ml lipofectamine 2000 in OptiMem (both Invitrogen) over 4-6 hr one day after plating at 50,000 cells/cm².

Extracts were assessed for RASSF1A protein and mRNA, which could only be detected up to 72 hr after transfection and only if cells were not replated; no stable clones were obtained after three independent attempts. Light microscopy did not reveal more toxicity with RASSF1A transfection compared to empty vector (about 20% cell death for both, data not shown) suggesting that short term expression was related to only brief transient presence of vector in cells. Thus to further determine whether RASSF1A might participate in IFN-induced apoptosis, immunoblotting for RASSF1A was performed after IFNs and DNMT1 AS (FIG. 7A). IFN α or IFN β (50 U/ml over 48 hr) treatment of DNMT1 AS but not mismatch oligonucleotide pretreated ACHN cells led to increased RASSF1A protein (FIG. 7A). Transcription of RASSF1A, however, was not affected, as determined by semi-quantitative RT-PCR (FIG. 7B) suggesting posttranscriptional regulation of RASSF1A expression.

### EXAMPLE 6

### Sensitization to IFN-Induced Apoptosis

Cell death in response to IFNs has been described in a variety of malignant cell types as due to apoptosis. Apoptosis in response to IFNs with or without prior DNMT1 inhibition was therefore examined.

After the indicated time of DNMT1 inhibition cells were plated at 5000 cells/cm² for TUNEL assay with IFNs added 16 hr after plating. Four to 5 days after IFN administration cells were harvested and processed according to the manufacturer's instructions for TUNEL flow cytometric analysis (BD Pharmingen, San Diego, Calif.). Apoptosis was confirmed with an assay for the activity of caspase 3 (BD Clontech, Palo Alto, Calif.), performed according to the manufacturers instructions, or caspase 3 cleavage detection by immunoblot (polyclonal caspase 3 antibody from Biomol) forty-eight hr after IFN treatment.

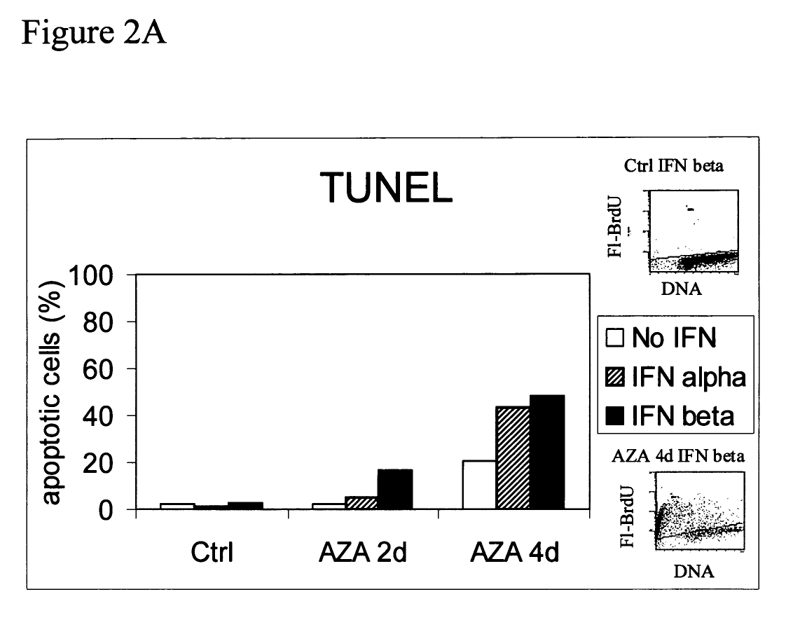

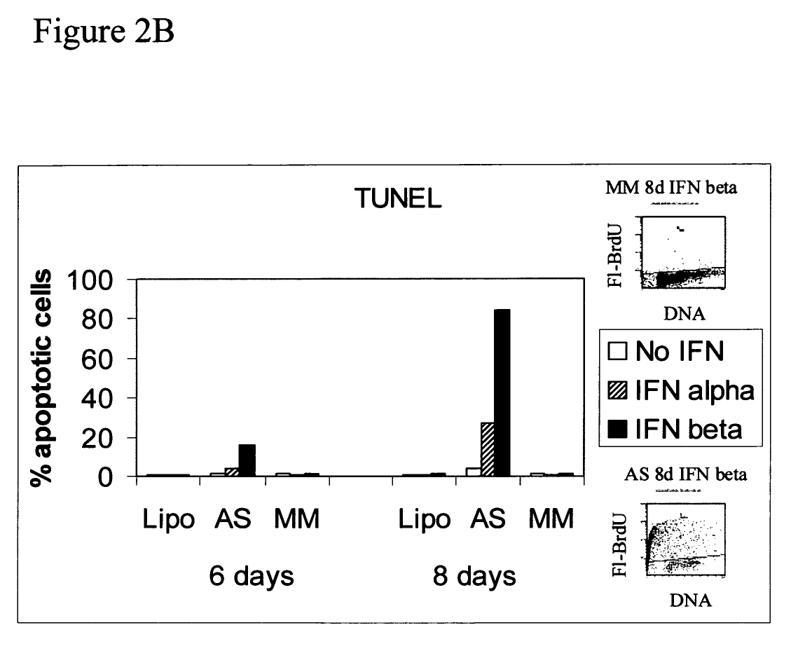

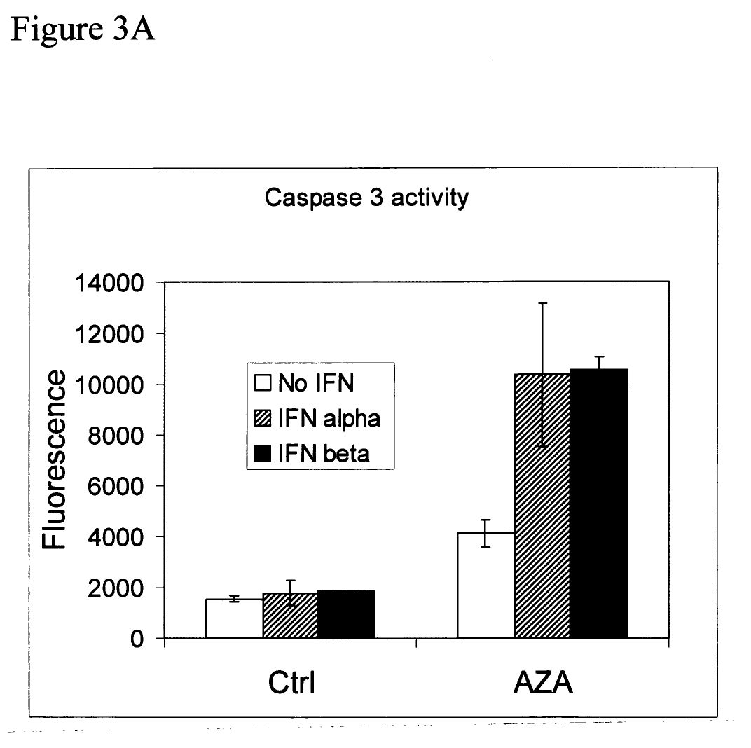

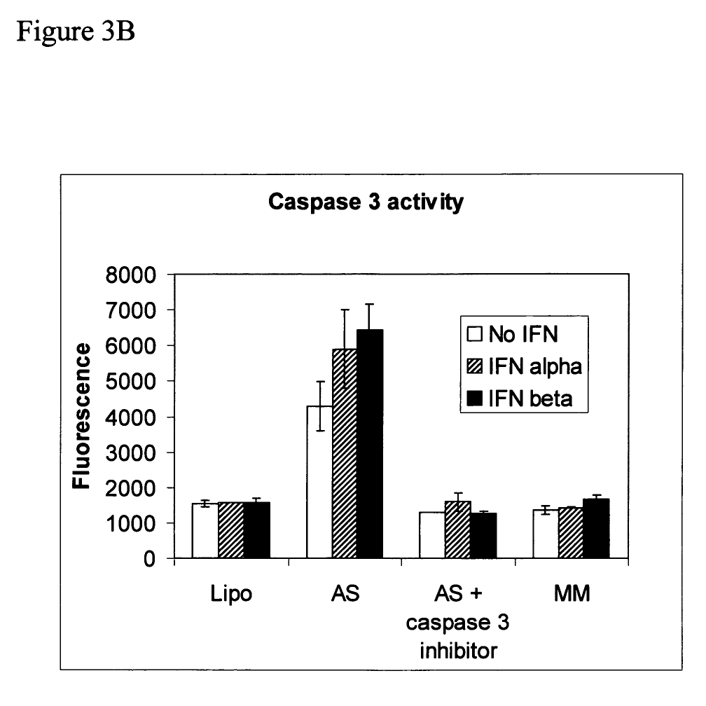

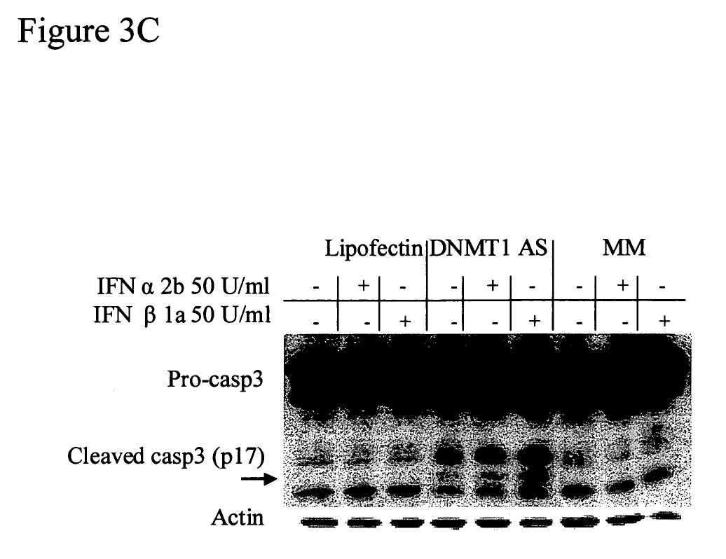

The duration and degree of DNMT1 protein suppression was found to be similar in response to either DNMT1 AS or 5-AZA-dC treatments of ACHN cells (FIG. 1). ACHN cells were resistant to up to 500 U/ml IFN alpha 2b or beta 1a (<5% TUNEL positive after 5 days) (data not shown), however treatment with 5-AZA-dC over 24 days or transfection with DNMT1 AS but not with mismatch control oligonucleotide over 6 to 8 days led to marked apoptosis (up to 50-80% cells apoptotic on TUNEL staining) 4-5 days after low dose (50 U/ml) IFN α or IFN β were applied (FIGS. 2A-B). While 4 days of 5-AZA-dC treatment caused some apoptosis (20% TUNEL positive), treatment with DNMT1 AS alone or MM alone did not (TUNEL <5%, FIG. 2A-B). These results demonstrate that inhibition of DNMT1 can enhance the sensitivity of human renal cancer cells to IFN.

### EXAMPLE 7

### Treatment of Human Patients Having RCC

This is a two-part study. The first stage is a dose and schedule-optimizing study of MG98 given as either an intermittent or continuous intravenous (IV) infusion in combination with INF. The second stage is a randomized efficacy evaluation of the combination of MG98 administered in the selected schedule with INF compared to treatment with INF alone.

In the first stage of the study, patients are assigned to one of two schedules of MG98. In one group, patients are treated with a 2-hour IV infusion of MG98 given twice per week for three weeks followed by one week of rest. This is administered with a separate fixed dose of INF administered subcutaneously three times per week over the full course of treatment. In the second group, patients receive MG98 in two 7-day continuous IV infusions, each followed by one week of rest and combined with the same dose and schedule of INF as the first group. Both 4-week regimens constitute 1 cycle of treatment. In each schedule, two out of three pre-selected dose levels of MG98 are administered to patients in combination with the fixed dose of INF. The MG98 starting dose (N) in each schedule is an intermediate dose level. Patients are enrolled in cohorts of 3. Toxicity assessments are used to guide the number of patients treated (3 or 6) and to decide whether the second MG98 dose level in each schedule is higher (N+1) or lower (N−1) than the starting dose. In each schedule, the cohort of patients treated at the highest MG98 dose that is adequately tolerated in combination with INF is expanded to 9 patients (total). When these 9 patients have completed 2 cycles of treatment, a comparison of toxicity and early progression is conducted in order to select one of the two schedules for the second stage of the study. Pharmacokinetic evaluations of MG98 and INF along with evaluation of DNMT1 mRNA suppression in PBMC's are conducted during cycle 1 in all patients in this portion of the study.

In the second stage of the study, patients are randomized with equal probability either to treatment with the recommended dose and schedule of MG98 combined with INF or to treatment with the same dose and schedule of INF administered as a single agent. The primary objective of the study is to compare the progression free survival of patients in the two groups. The safety and tolerability of the two regimens is assessed. Pharmacokinetic evaluations of MG98 and IFN are conducted in a limited number of patients (approximately 20). PBMC's are also collected in order to assess the degree of suppression of DNMT1 mRNA in patients treated with MG98. In the first stage of this study, two schedules of MG98 administration are combined with INF. Within each schedule two out of a possible three pre-selected doses of MG98 are evaluated in order to identify the best tolerated dose for combination with INF in each schedule. Based on clinical experience to date and the potential for overlapping toxicities when MG98 is combined with INF, a starting dose of 160 mg/m²/day of MG98 has been selected for the intermittent schedule. This dose is 44% below the dose where grade 3 transaminitis was seen in nephrectomized patients thus leaving ample room for any potential increased toxicity due to the concomitant administration of INF. Investigation of a continuous intravenous infusion schedule for administration of MG98 is based on tumour xenograft models showing that daily dosing of MG98 produced the greatest anti-tumour activity compared to the intermittent administration. Three Phase I studies using continuous infusion schedules have been conducted. A Phase I study where MG98 was given as a 21-day infusion resulted in frequent adverse events at dose levels above 80 mg/m²/day. In two other Phase I studies, MG98 has been administered as a 2 hour infusion followed by a 5 day continuous infusion and as a 7 day and 14 day infusion. Doses up to 200 mg/m²/day have been given in these schedules. However, given the potential for increased toxicity in nephrectomized patients and the concomitant administration of INF, a starting dose of 125 mg/m²/day has been selected for this schedule since this is known to be tolerated over the duration of infusion for this trial. In view of the preclinical data that demonstrated a potential benefit of pre-treating INF resistant cell lines with MG98, all patients have their INF treatment held for the first week of treatment in the first cycle of treatment only. This allows patients receiving combination treatment to receive MG98 alone prior to initiation of their treatment with INF. The same schedule (INF starting on Day 8) applies to patients receiving INF alone for purposes of consistency. In general, the therapeutic efficacy of INF monotherapy is similar with adapted dosing intervals as long as the weekly total doses are between 25 to 45 MIU. Based on this objective, the starting dose of INF administered in combination with MG98 or as monotherapy is about 12 MIU/m²/day given subcutaneously three times per week throughout the course of treatment. All patients are treated and/or followed for at least 1 year.

Eighteen (18) patients have been treated with the MG98 and interferon combination, 9 on an intermittent dosing schedule and 9 on a continuous dosing schedule. Four patients have shown favourable activity: two on each regimen, of which, three of these four patients experienced partial responses (one confirmed according to formal RECIST criteria), and the fourth patient had symptomatic improvement of bone disease including prolonged stable disease.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A method for sensitizing an interferon (IFN)-resistant cell to IFN-induced apoptosis comprising demethylating a gene of the IFN-resistant cell effecting IFN resistance.

2. The method according to claim 1, comprising contacting the cell with an agent that reduces expression and/or activity of a DNA methyltransferase (DNMT).

3. The method according to claim 2, wherein the agent is a small molecule inhibitor of a DNMT

4. The method according to claim 3, wherein the small molecule inhibitor is 5-AZA-dC.

5. The method according to claim 3, wherein the small molecule inhibitor is selected from the group consisting of 5-aza-cytidine (5-AZA-C), 5-fluoro-2′-deoxycytidine, 5,6-dihydro-5-azacytidine and Zebularine.

6. The method according to claim 2, wherein the agent is an antisense oligonucleotide complementary to a DNMT mRNA.

7. The method according to claim 3, wherein the antisense oligonucleotide comprises the sequence of SEQ ID NO:2, or a derivative thereof.

8. The method according to claim 3, wherein the antisense oligonucleotide comprises a sequence selected from the group consisting of SEQ ID NO:1, and SEQ ID NO:3-39, or a derivative thereof.

9. The method according to claim 2, wherein reducing expression and/or activity of a DNMT demethylates the gene.

10. The method according to claim 1, wherein demethylating the gene activates the gene.

11. The method according to claim 1, wherein the gene is a silenced gene.

12. The method according to claim 11, wherein the gene is RASSF1A.

13. The method according to claim 12, wherein RASSF1A protein expression is increased.

14. The method according to claim 1, wherein the cell is a cancer cell.

15. The method according to claim 14, wherein the cancer is renal cell carcinoma.

16. The method according to claim 14, wherein the cancer is a melanoma.

17. The method according to claim 2, wherein the DNMT is DNMT-1.

18. A method of inducing apoptosis in an IFN-resistant cell comprising:
a) sensitizing the cell to IFN-induced apoptosis; and
b) contacting the cell with an IFN.

19. The method of claim 18, wherein step a) is effected by demethylating a gene of the IFN-resistant cell effecting IFN resistance.

20. The method of claim 19, wherein demethylating the gene is effected by contacting the cell with an agent that reduces expression and/or activity of a DNMT.

21. The method according to claim 20 wherein the agent is a small molecule inhibitor of a DNMT

22. The method according to claim 21, wherein the small molecule inhibitor is 5-AZA-dC.

23. The method according to claim 21, wherein the small molecule inhibitor is selected from the group consisting of 5-aza-cytidine (5-AZA-C), 5-fluoro-2′-deoxycytidine, 5,6-dihydro-5-azacytidine and Zebularine.

24. The method according to claim 20, wherein the agent is an antisense oligonucleotide complementary to a DNMT mRNA.

25. The method according to claim 24 wherein the antisense oligonucleotide comprises the sequence of SEQ ID NO:2, or a derivative thereof.

26. The method according to claim 24, wherein the antisense oligonucleotide comprises a sequence selected from the group consisting of SEQ ID NO:1, and SEQ ID NO:3-39, or a derivative thereof.

27. The method according to claim 20, wherein reducing expression and/or activity of a DNMT demethylates the gene.

28. The method according to claim 19, wherein demethylating the gene activates the gene.

29. The method according to claim 19, wherein the gene is a silenced gene.

30. The method according to claim 29, wherein the gene is RASSF1A.

31. The method according to claim 30, wherein RASSF1A protein expression is increased.

32. The method according to claim 18, wherein the cell is a cancer cell.

33. The method according to claim 32, wherein the cancer is renal cell carcinoma.

34. The method according to claim 32, wherein the cancer is a melanoma.

35. The method according to claim 20, wherein the DNMT is DNMT-1.

36. The method of claim 18, wherein the IFN is selected from the group consisting of IFN-α, IFN-β and combinations thereof.

37. The method of claim 18, wherein step b) is effected by contacting the cell with an IFN-inducing agent, wherein the IFN-inducing agent causes the cell to produce an endogenous or recombinant IFN.

38. The method of claim 37, wherein the IFN-inducing agent is an agent selected from the group consisting of poly-I:C, a double-stranded RNA and an immunostimulatory oligonucleotide.

39. The method of claim 18, wherein step a) and step b) are performed sequentially.

40. The method of claim 18, wherein step a) and step b) are performed concurrently.

41. A method for treating a cancer patient having an IFN-resistant cancer cell comprising:
a) sensitizing the IFN-resistant cancer cell to IFN-induced apoptosis; and
b) contacting the cell with a treatment effective amount of an IFN.

42. The method according to claim 41, wherein sensitizing the IFN-resistant cancer cell to IFN-induced apoptosis comprises demethylating a gene of the IFN-resistant cancer cell effecting IFN resistance.

43. The method according to claim 42, wherein demethylating the gene is effected by administering to the patient a treatment effective amount of an agent that reduces expression and/or activity of a DNMT.

44. The method according to claim 43 wherein the agent is a small molecule inhibitor of a DNMT.

45. The method according to claim 44, wherein the small molecule inhibitor is 5-AZA-dC.

46. The method according to claim 44, wherein the small molecule inhibitor is selected from the group consisting of 5-aza-cytidine (5-AZA-C), 5-fluoro-2′-deoxycytidine, 5,6-dihydro-5-azacytidine and Zebularine.

47. The method according to claim 43, wherein the agent is an antisense oligonucleotide complementary to a DNMT mRNA.

48. The method according to claim 47 wherein the antisense oligonucleotide comprises the sequence of SEQ ID NO:2, or a derivative thereof.

49. The method according to claim 47, wherein the antisense oligonucleotide comprises a sequence selected from the group consisting of SEQ ID NO:1, and SEQ ID NO:3-39, or a derivative thereof.

50. The method according to claim 43, wherein reducing expression and/or activity of a DNMT demethylates the gene.

51. The method according to claim 42, wherein demethylating the gene activates the gene.

52. The method according to claim 51, wherein the gene is a silenced gene.

53. The method according to claim 52, wherein the gene is RASSF1A.

54. The method according to claim 53, wherein RASSF1A protein expression is increased.

55. The method according to claim 41, wherein the cancer is renal cell carcinoma and the IFN-resistant cancer cell is a cell thereof.

56. The method according to claim 41, wherein the cancer is a cancer selected from the group consisting of melanoma and the IFN-resistant cancer cell is a cell thereof.

57. The method according to claim 43, wherein the DNMT is DNMT-1.

58. The method of claim 41, wherein the IFN is selected from the group consisting of IFN-α, IFN-β and combinations thereof.

59. The method of claim 41, wherein step b) is effected by administering to the patient a treatment effective amount of an IFN selected from the group consisting of IFN-α, IFN-β and a combination thereof.

60. The method of claim 41, wherein step b) is effected by administering to the patient an IFN-inducing effective amount of an IFN-inducing agent, wherein the IFN-inducing agent causes the cell to produce an endogenous or recombinant IFN.

61. The method of claim 60, wherein the IFN-inducing agent is an agent selected from the group consisting of poly-I:C, a double-stranded RNA and an immunostimulatory oligonucleotide.

62. The method of claim 41, wherein step a) and step b) are performed sequentially.

63. The method of claim 41, wherein step a) and step b) are performed concurrently.


SEQUENCE LISTING (50 sequences)

SEQ ID NO: 1 - DNA, 20 bp, Artificial Sequence
  Description of Artificial Sequence Synthetic oligonucleotide
aagcatgagcaccgttctcc

SEQ ID NO: 2 - DNA, 20 bp, Artificial Sequence
  Description of Artificial Sequence Synthetic oligonucleotide
ttcatgtcagccaaggccac

SEQ ID NO: 3 - DNA, 20 bp, Artificial Sequence
  Description of Artificial Sequence Synthetic oligonucleotide
gctgtctctttccaaatctt

SEQ ID NO: 4 - DNA, 20 bp, Artificial Sequence
  Description of Artificial Sequence Synthetic oligonucleotide
tttctgttaagctgtctctt

SEQ ID NO: 5 - DNA, 20 bp, Artificial Sequence
  Description of Artificial Sequence Synthetic oligonucleotide
ttctccttcacacattcctt

SEQ ID NO: 6 - DNA, 20 bp, Artificial Sequence
  Description of Artificial Sequence Synthetic oligonucleotide
cgtgcaagagattcaatttc

SEQ ID NO: 7 - DNA, 20 bp, Artificial Sequence
  Description of Artificial Sequence Synthetic oligonucleotide
aagtcacataactgattctt

SEQ ID NO: 8 - DNA, 20 bp, Artificial Sequence
  Description of Artificial Sequence Synthetic oligonucleotide
ctcggataattcttctttac

SEQ ID NO: 9 - DNA, 20 bp, Artificial Sequence
  Description of Artificial Sequence Synthetic oligonucleotide
ccaggtagccctcctcggat

SEQ ID NO: 10 - DNA, 20 bp, Artificial Sequence
  Description of Artificial Sequence Synthetic oligonucleotide
agggatttgactttagccag

SEQ ID NO: 11 - DNA, 20 bp, Artificial Sequence
  Description of Artificial Sequence Synthetic oligonucleotide
tccaaggacaaatctttatt

SEQ ID NO: 12 - DNA, 20 bp, Artificial Sequence
  Description of Artificial Sequence Synthetic oligonucleotide
catgagcaccgttctccaag

SEQ ID NO: 13 - DNA, 20 bp, Artificial Sequence
  Description of Artificial Sequence Synthetic oligonucleotide
acgtccattcacttcccggt

SEQ ID NO: 14 - DNA, 20 bp, Artificial Sequence
  Description of Artificial Sequence Synthetic oligonucleotide
tcacttcttgcttgcttccc

SEQ ID NO: 15 - DNA, 20 bp, Artificial Sequence
  Description of Artificial Sequence Synthetic oligonucleotide
gcttggttcccgttttctag

SEQ ID NO: 16 - DNA, 20 bp, Artificial Sequence
  Description of Artificial Sequence Synthetic oligonucleotide
ctagacgtccattcacttcc

SEQ ID NO: 17 - DNA, 20 bp, Artificial Sequence
  Description of Artificial Sequence Synthetic oligonucleotide
actctacgggcttcacttct

SEQ ID NO: 18 - DNA, 20 bp, Artificial Sequence
  Description of Artificial Sequence Synthetic oligonucleotide
tctgccattcccactctacg

SEQ ID NO: 19 - DNA, 20 bp, Artificial Sequence
  Description of Artificial Sequence Synthetic oligonucleotide
catctgccattcccactcta

SEQ ID NO: 20 - DNA, 20 bp, Artificial Sequence
  Description of Artificial Sequence Synthetic oligonucleotide
ggcatctgccattcccactc

SEQ ID NO: 21 - DNA, 20 bp, Artificial Sequence
  Description of Artificial Sequence Synthetic oligonucleotide
atcggacttgctcctcctgg

SEQ ID NO: 22 - DNA, 20 bp, Artificial Sequence
  Description of Artificial Sequence Synthetic oligonucleotide
ggtgacgggagggcagaact

SEQ ID NO: 23 - DNA, 20 bp, Artificial Sequence
  Description of Artificial Sequence Synthetic oligonucleotide
tgccagaaacaggggtgacg

SEQ ID NO: 24 - DNA, 20 bp, Artificial Sequence
  Description of Artificial Sequence Synthetic oligonucleotide
gtgcatgttggggattcctg

SEQ ID NO: 25 - DNA, 20 bp, Artificial Sequence
  Description of Artificial Sequence Synthetic oligonucleotide
gtgaacggacagattgacat

SEQ ID NO: 26 - DNA, 20 bp, Artificial Sequence
  Description of Artificial Sequence Synthetic oligonucleotide
aggccacaaacaccatgtac

SEQ ID NO: 27 - DNA, 20 bp, Artificial Sequence
  Description of Artificial Sequence Synthetic oligonucleotide
cgaacctcacacaacagctt

SEQ ID NO: 28 - DNA, 20 bp, Artificial Sequence
  Description of Artificial Sequence Synthetic oligonucleotide
gataagcgaacctcacacaa

SEQ ID NO: 29 - DNA, 20 bp, Artificial Sequence
  Description of Artificial Sequence Synthetic oligonucleotide
ctgcacaatttgatcactaa

SEQ ID NO: 30 - DNA, 20 bp, Artificial Sequence
  Description of Artificial Sequence Synthetic oligonucleotide
cagaaacaggggtgacggga

SEQ ID NO: 31 - DNA, 20 bp, Artificial Sequence
  Description of Artificial Sequence Synthetic oligonucleotide
gcacaaagtactgcacaatt

SEQ ID NO: 32 - DNA, 20 bp, Artificial Sequence
  Description of Artificial Sequence Synthetic oligonucleotide
tccagaatgcacaaagtact

SEQ ID NO: 33 - DNA, 20 bp, Artificial Sequence
  Description of Artificial Sequence Synthetic oligonucleotide
ccaaggccacaaacaccatg

SEQ ID NO: 34 - DNA, 20 bp, Artificial Sequence
  Description of Combined DNA/RNA Molecule Synthetic oligonucleotide
ccaggtagccctcctcggau

SEQ ID NO: 35 - DNA, 20 bp, Artificial Sequence
  Description of Combined DNA/RNA Molecule Synthetic oligonucleotide
aagcatgagcaccgttcucc

SEQ ID NO: 36 - DNA, 20 bp, Artificial Sequence
  Description of Combined DNA/RNA Molecule Synthetic oligonucleotide
uucatgtcagccaaggccac

SEQ ID NO: 37 - DNA, 20 bp, Artificial Sequence
  Description of Combined DNA/RNA Molecule Synthetic oligonucleotide
cgaacctcacacaacagcuu

SEQ ID NO: 38 - DNA, 20 bp, Artificial Sequence
  Description of Combined DNA/RNA Molecule Synthetic oligonucleotide
gauaagcgaacctcacacaa

SEQ ID NO: 39 - DNA, 20 bp, Artificial Sequence
  Description of Combined DNA/RNA Molecule Synthetic oligonucleotide
ccaaggccacaaacaccaug

SEQ ID NO: 40 - DNA, 20 bp, Artificial Sequence
  Description of Artificial Sequence Synthetic oligonucleotide
ttaatgtaacctaaggtcaa

SEQ ID NO: 41 - DNA, 18 bp, Artificial Sequence
  Description of Artificial Sequence Synthetic oligonucleotide
gggttttgcgagagcgcg

SEQ ID NO: 42 - DNA, 18 bp, Artificial Sequence
  Description of Artificial Sequence Synthetic oligonucleotide
gccaagcgcaaacaatcg

SEQ ID NO: 43 - DNA, 22 bp, Artificial Sequence
  Description of Artificial Sequence Synthetic oligonucleotide
ggttttgtgagagtgtgtttag

SEQ ID NO: 44 - DNA, 21 bp, Artificial Sequence
  Description of Artificial Sequence Synthetic oligonucleotide
aaaccaaacacaaacaatcac

SEQ ID NO: 45 - DNA, 22 bp, Artificial Sequence
  Description of Artificial Sequence Synthetic oligonucleotide
agcgtgccaacgcgctgcgcat

SEQ ID NO: 46 - DNA, 22 bp, Artificial Sequence
  Description of Artificial Sequence Synthetic oligonucleotide
caggctcgtccacgttcgtgtc

SEQ ID NO: 47 - DNA, 19 bp, Artificial Sequence
  Description of Artificial Sequence Synthetic oligonucleotide
cagacctactcagggattc

SEQ ID NO: 48 - DNA, 19 bp, Artificial Sequence
  Description of Artificial Sequence Synthetic oligonucleotide
gagccagacgctgctttgt

SEQ ID NO: 49 - DNA, 18 bp, Artificial Sequence
  Description of Artificial Sequence Synthetic oligonucleotide
cgcccagtctggatcctg

SEQ ID NO: 50 - DNA, 21 bp, Artificial Sequence
  Description of Artificial Sequence Synthetic oligonucleotide
ctcaatgcctgccttattctg